(19) United States
(12) Patent Application Publication
(10) Pub. No.: US 2019/0209031 A1
(43) Pub. Date: Jul. 11, 2019

(54) BLOOD PRESSURE MEASURING DEVICE, BLOOD PRESSURE MEASURING METHOD AND RECORDING MEDIUM HAVING BLOOD PRESSURE MEASURING PROGRAM RECORDED THEREIN

(71) Applicant: NEC Corporation, Minato-ku, Tokyo (JP)

(72) Inventors: Tetsuri ARIYAMA, Tokyo (JP); Katsumi ABE, Tokyo (JP); Masahiro KUBO, Tokyo (JP); Yuji OHNO, Tokyo (JP); Takeshi AKAGAWA, Tokyo (JP)

(73) Assignee: NEC Corporation, Minato-ku, Tokyo (JP)

(21) Appl. No.: 16/328,806

(22) PCT Filed: Sep. 1, 2017
(86) PCT No.: PCT/JP2017/031522

§ 371 Date: Feb. 27, 2019

(30) Foreign Application Priority Data
Sep. 5, 2016 (JP) 2016-172555

(51) Int. Cl. A61B 5/021; A61B 5/0456; A61B 5/00

(52) U.S. Cl. CPC A61B 5/02125; A61B 5/0456; A61B 5/681; A61B 5/7282; A61B 5/0082

(57) ABSTRACT

This blood pressure measuring device includes: first and second electrodes which contact the body surface near an artery; an electrocardiogram measuring means which measures a potential difference between the first electrode and the second electrode and obtains a first time at which at least a prescribed portion is generated in an electrocardiogram; a pulse wave detecting means which detects pulse wave information from the body surface near the artery; a pulse wave measuring means which obtains, from the pulse wave information, a second time at which a prescribed portion is generated in the pulse wave; and a blood pressure estimating means which calculates a pulse wave propagation time from the first time and the second time, and calculates an estimated blood pressure on the basis of a relationship between the pulse wave propagation time, a predefined pulse wave propagation time, and a blood pressure value.

### TECHNICAL FIELD

The present invention relates to a blood pressure measuring device, a blood pressure measuring method, and a recording medium having a blood pressure measuring program recorded therein.

### BACKGROUND ART

At present, in order to measure blood pressure of a person, there is used a method in which an air bag called a cuff is wound around an upper arm, air is supplied to the cuff, the upper arm is thereby pressed, and blood pressure is estimated from a pressurized pulse wave acquired thereby. However, since this method gives a large burden to a user, there is proposed a method in which blood pressure is estimated by using a correlation between blood pressure and time called pulse wave transit time spent by a pressure wave (pulse wave) accompanying contraction of a heart to transit through a blood vessel. For blood pressure measurement that uses pulse wave transit time, an electrocardiogram and a pulse wave need to be measured, and a plurality of sensors need to be stuck to a plurality of regions of a body. In view of the above, PTL 1 proposes a method in which, in order to measure an electrocardiogram and a pulse wave, a wristwatch-type device is attached to one arm, a fingertip of the other arm is brought into contact with a sensor of the wristwatch-type device, and thereby blood pressure is measured.

Generally, when an electrocardiogram is measured, electrodes need to be stuck on a plurality of regions such as four limbs and a chest. PTL 2 describes a technique of electrocardiogram measurement at an upper arm, as an invention for measurement from one certain region of a body. In the technique described in PTL 2, an electrode array and an electrode R are stuck on an upper arm, and from a plurality of the electrodes, the maximum electrocardiogram signal is acquired.

PTL 3 also describes a blood pressure measuring device that estimates blood pressure from a relation formula between pulse wave transit time or a pulse wave transit speed and a blood pressure value. In this device, electrocardiographic electrodes are attached to one arm and the other arm of a body, respectively, and a photoelectric sensor is attached to a finger of the other. The two electrocardiographic electrodes and the photoelectric sensor constitute a slave unit, and a cuff and a control circuit constitute a master unit. For measurement, the cuff wound around the arm is first pressurized. When the pressurization is completed, an R wave for an electrocardiogram is detected by the two electrocardiographic electrodes, and a peak time tR at the time of the detection is recorded. Further, cuff pressure at that time is detected. Furthermore, a rise of a photoelectric pulse wave at a fingertip portion is detected, and a rise time tS is recorded. By using a difference between tR and tS, blood pressure is calculated (paragraphs (0010) and (0018), FIG. 3 and the like). In addition, PTL 4 describes an electronic wristwatch-type sphygmomanometer. This sphygmomanometer is attached to a wrist, and a fingertip of a hand opposite to the hand of the attachment is put on an electrocardiographic wave detection electrode. In this state, an electrocardiographic wave detection control unit detects an electrocardiographic wave (R wave) from a potential difference between a detected potential of the electrocardiographic wave detection electrode and a detected potential of a back cover. Further, the sphygmomanometer includes an optical element unit provided with a light emission diode (LED) and a phototransistor. Light emitted from the LED is reflected by the fingertip, and the reflected light is input to the phototransistor and photoelectrically converted. A signal acquired by the photoelectric conversion indicates a pulse. From a time difference in detection timing between the electrocardiographic wave and the pulse, a blood pressure value is calculated.

### CITATION LIST

### Patent Literature

[PTL 1] Japanese Patent No. 3785529

[PTL 2] Japanese Patent No. 5428889

[PTL 3] Japanese Unexamined Patent Application Publication No. H7-308295

[PTL 4] Japanese Unexamined Patent Application Publication No. H7-116141

### SUMMARY OF INVENTION

### Technical Problem

In the technique described in above PTL 1, freedom of both hands is deprived in measuring an electrocardiogram and a pulse wave necessary for blood pressure measurement. Further, since a cuff is attached to an upper arm as a calibration, separately from a wristwatch-type device, and blood pressure is then measured, there is a problem that a configuration of an entire device becomes more troublesome.

Further, in the technique described in above PTL 2, since many electrodes are required, it cannot be said that a burden on a user is reduced as compared with a general electrocardiogram measuring device. In addition, array electrodes and an electrode R need to be separated from each other and be made to contact with a living body, which is troublesome.

Furthermore, in PTL 3, electrocardiographic electrodes are attached respectively to two arms, and thus the attachment is troublesome, and both hands are restrained during measurement.

In addition, in PTL 4, a sphygmomanometer needs to be attached to one arm, and a fingertip of an opposite arm needs to be put on an optical element unit of the sphygmomanometer, and thus both hands are restrained during measurement.

### OBJECT OF INVENTION

An object of the present invention is to provide a blood pressure measuring device, a blood pressure measuring method, and a recording medium having a blood pressure measuring program recorded therein, whereby the above-described problem is solved, and blood pressure measurement is enabled simply by attaching electrodes to one region of a body.

### Solution to Problem

An aspect of the present invention is a blood pressure measuring device comprising: a first electrode and a second electrode that are made to contact with a body surface near an artery; electrocardiogram measuring means for measuring a potential difference between the first electrode and the second electrode, and acquiring a first time at which at least a predetermined portion in an electrocardiogram occurs; pulse wave detecting means for detecting pulse wave information from a body surface near the artery; pulse wave measuring means for acquiring, from the pulse wave information, a second time at which a predetermined portion in a pulse wave occurs; and blood pressure estimating means for calculating pulse wave transit time from the first time and the second time, and calculating estimated blood pressure, based on the pulse wave transit time and a predefined relation between pulse wave transit time and a blood pressure value.

Another aspect of the present invention is a blood pressure measuring method comprising: making a first electrode and a second electrode in contact with a body surface near an artery; measuring a potential difference between the first electrode and the second electrode, and acquiring a first time at which at least a predetermined portion in an electrocardiogram occurs; detecting pulse wave information from a body surface near the artery; acquiring, from the pulse wave information, a second time at which a predetermined portion in the pulse wave occurs; and calculating pulse wave transit time from the first time and the second time, and calculating estimated blood pressure, based on the pulse wave transit time and a predefined relation between pulse wave transit time and a blood pressure value.

Still another aspect of the present invention is a recording medium recording a blood pressure measuring program that causes a computer to execute: processing of measuring a potential difference between a first electrode and a second electrode made to contact with a body surface near an artery, and acquiring a first time at which at least a predetermined portion in an electrocardiogram occurs; processing of detecting pulse wave information from a body surface near the artery; processing of acquiring, from the pulse wave information, a second time at which a predetermined portion in the pulse wave occurs; and processing of calculating pulse wave transit time from the first time and the second time, and calculating estimated blood pressure, based on the pulse wave transit time and a predefined relation between pulse wave transit time and a blood pressure value.

### Advantageous Effect of Invention

According to the present invention, blood pressure can be measured simply by attaching electrodes to one region of a body

### BRIEF DESCRIPTION OF DRAWINGS

FIG. 1 is a block diagram of a blood pressure measuring device according to a first example embodiment of the present invention.

FIG. 2 is a plan view and a cross-sectional view of a blood pressure measuring device according to the first example embodiment of the present invention.

FIG. 3 is a diagram illustrating a basic waveform of an electrocardiogram.

FIG. 4 is a diagram illustrating a basic waveform of a pulse wave.

FIG. 5 is a diagram illustrating pulse wave transit time that is a time difference between a peak of an R wave of an electrocardiogram and a rise of a pulse wave.

FIG. 6 is a schematic view of attachment of the blood pressure measuring device according to the first example embodiment of the present invention.

FIG. 7 is an image diagram illustrating that a polarity of an electrocardiogram waveform is inverted by a first electrode and a second electrode crossing over an artery.

FIG. 8 is a diagram illustrating an electrocardiogram measurement result in the first example embodiment according to the present invention and an electrocardiogram measurement result in an existing method.

FIG. 9 is a block diagram of a blood pressure measuring device according to a second example embodiment of the present invention.

FIG. 10 is a plan view and a cross-sectional view of the blood pressure measuring device according to the second example embodiment of the present invention.

FIG. 11 is a schematic view of attachment of the blood pressure measuring device according to the second example embodiment of the present invention.

FIG. 12 is a block diagram of a blood pressure measuring device according to a third example embodiment of the present invention.

FIG. 13 is a plan view and a cross-sectional view of the blood pressure measuring device according to the third example embodiment of the present invention.

FIG. 14 is a schematic view of attachment of the blood pressure measuring device according to the third example embodiment of the present invention.

FIG. 15 is a block diagram of a blood pressure measuring device according to a fourth example embodiment of the present invention.

### EXAMPLE EMBODIMENT

Example embodiments of the present invention are described in detail below with reference to the drawings. Note that the present invention is not limited to the following example embodiments.

### First Example Embodiment

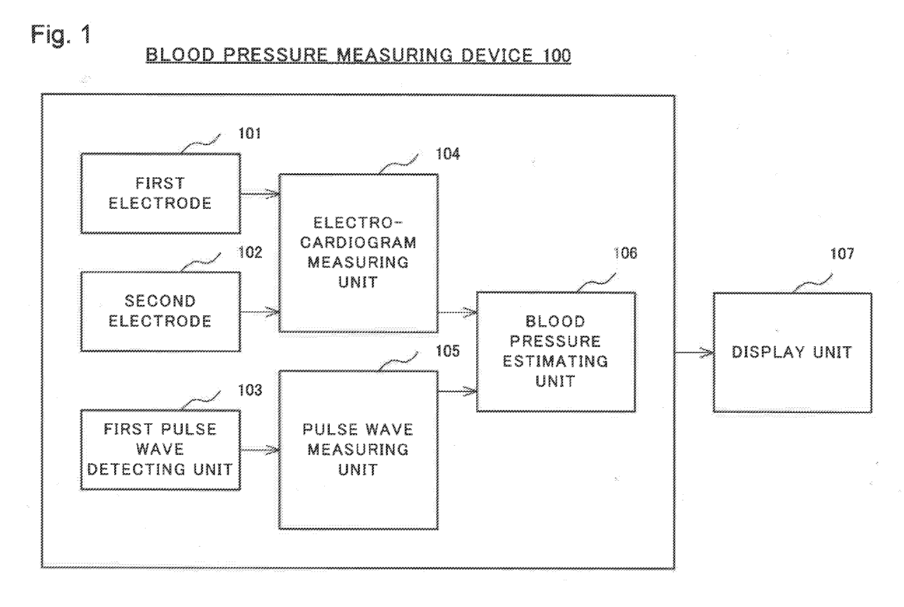

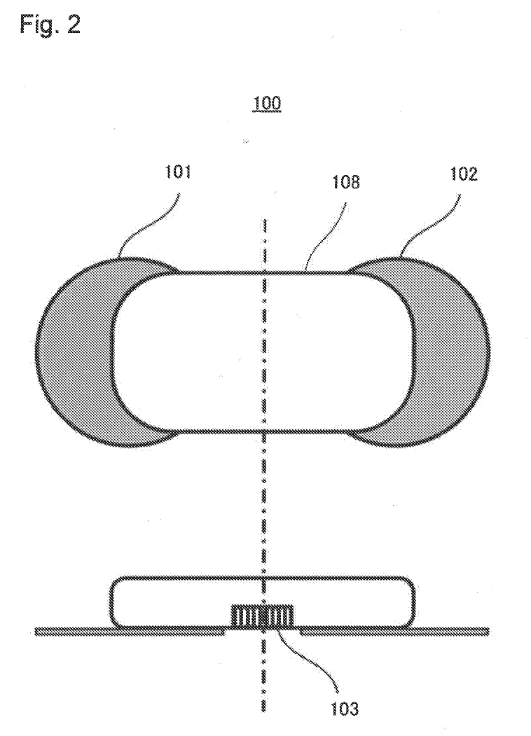

FIG. 1 is a block diagram of a blood pressure measuring device 100 according to a first example embodiment of the present invention. The blood pressure measuring device 100 includes a first electrode 101, a second electrode 102, a first pulse wave detecting unit 103, an electrocardiogram measuring unit 104, a pulse wave measuring unit 105, and a blood pressure estimating unit 106. FIG. 2 illustrates a plan view and a cross-sectional view of the blood pressure measuring device 100. The reference symbol 108 designates a casing of the blood pressure measuring device 100.

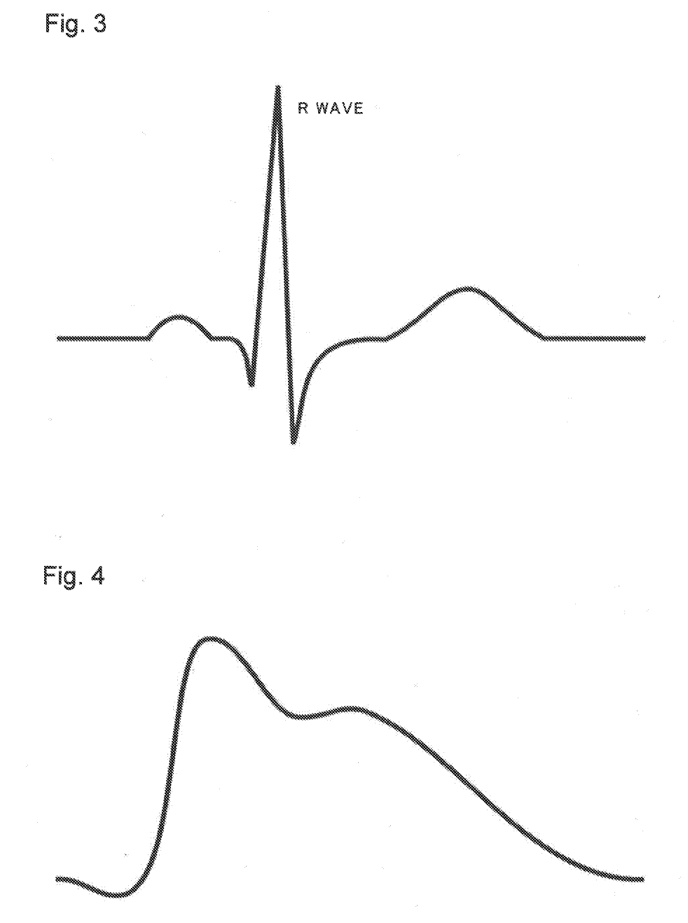

The first electrode 101 and the second electrode 102 are electrodes for acquiring an electrocardiogram that is a weak electric signal flowing through an entire body. The first electrode 101 and the second electrode 102 are provided at an end portion of the casing 108. Surfaces that belong to the first electrode 101 and the second electrode 102 and that contact with a body surface have an adhesive property, and enables the blood pressure measuring device 100 to be stuck to any position on a body surface of a person. Note that in the present example embodiment, planar shapes of the first electrode 101 and the second electrode 102 are circular. Although a shape of the casing 108 is roughly a rectangular parallelepiped in FIG. 2, it is desirable that the connection surfaces of the first electrode 101 and the second electrode 102 are somewhat curved with respect to an arrangement direction of the electrodes in such a way as to conform to a shape of an attachment region, resulting in closer contact with a body surface. The electrocardiogram measuring unit 104 calculates a potential difference between the first electrode 101 and the second electrode 102 that are in contact with a body surface, removes a body motion noise, a high-frequency noise, and the like, and acquires an electrocardiogram. FIG. 3 illustrates a basic waveform of an electrocardiogram.

The first pulse wave detecting unit 103 is positioned on a body surface side at a center between the first electrode 101 and the second electrode 102, and is constituted of any one of a vibration sensor, a pressure sensor, a piezoelectric sensor, an optical sensor, an ultrasonic sensor, a radio wave sensor, an electrostatic capacity sensor, an electric field sensor, and a magnetic field sensor. A plurality of types of these sensors or a plurality of these sensors may be used. The first pulse wave detecting unit 103 captures pulsation of an artery under a body surface under the first pulse wave detecting unit 103, and sends a signal such as vibration caused by the pulsation, to the pulse wave measuring unit 105. For example, when the first pulse wave detecting unit 103 is constituted of a light emission diode (LED) as a light emission element and a photo diode (PD) as a light reception element, light having any wavelength and generated by the LED is reflected inside a body, and the reflected light can be detected by the PD. An intensity of a signal detected by the PD has correlation with a quantity of blood flowing in a blood vessel, and thus, can be recognized as a pulse wave signal. FIG. 4 illustrates a basic waveform of a pulse wave. The pulse wave measuring unit 105 removes a body motion noise and a high-frequency noise included in the signal sent from the first pulse wave detecting unit 103, and extracts a pulse wave signal.

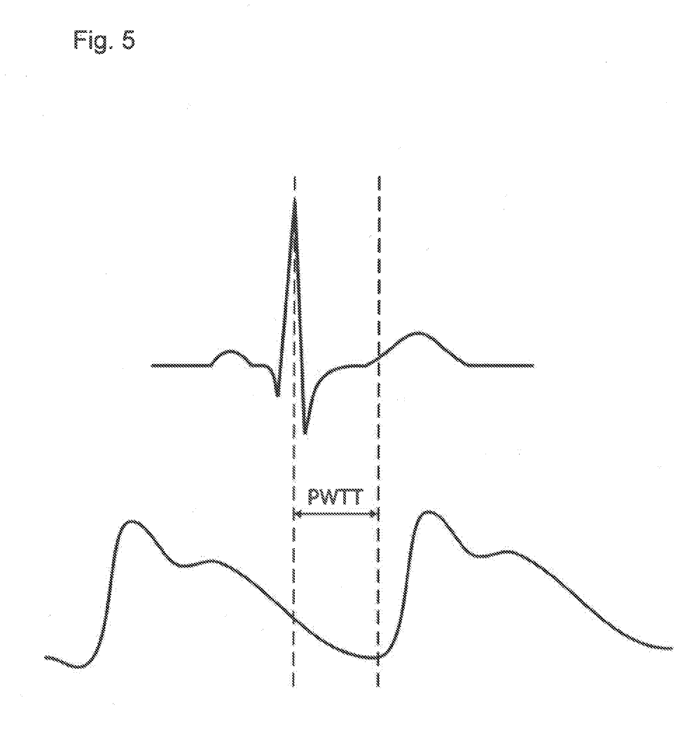

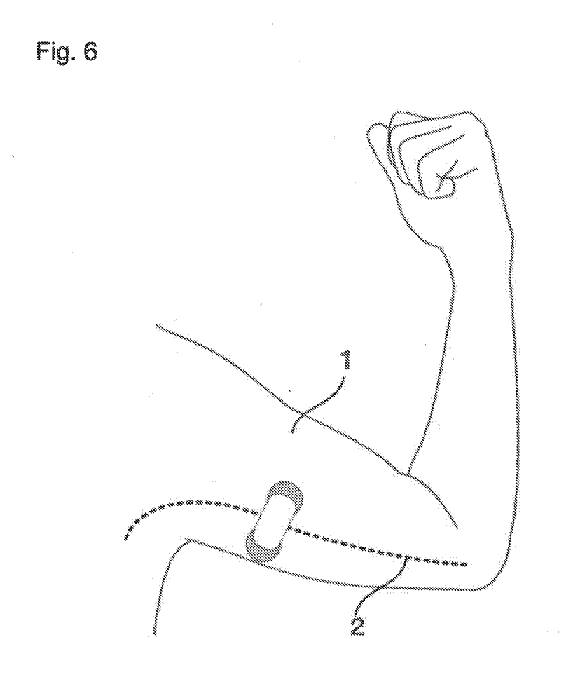

FIG. 5 illustrates an electrocardiogram, a pulse wave, and pulse wave transit time. The blood pressure estimating unit 106 calculates pulse wave transit time from a difference between peak time of an R wave of an electrocardiogram sent from the electrocardiogram measuring unit 104 and a rise time of a pulse wave sent from the pulse wave measuring unit 105. The blood pressure estimating unit 106 calculates an estimated value of systolic blood pressure from the calculated pulse wave transit time and a predefined relation formula between pulse wave transit time and a blood pressure value, and outputs the estimated value to the display unit 107. The relation formula for calculating an estimated value of systolic blood pressure is expressed in the following formula (1).

SBPest=α×PWTT+β Formula (1)

SBPest is an estimated value of systolic blood pressure, PWTT is the above-described pulse wave transit time, and α and β are parameters acquired in advance. There are mainly two methods for acquiring these α and β, i.e., a method that acquires pulse wave transit time and blood pressure value data from many subjects and is based on statistical analysis, and calibration, namely, a method that measures pulse wave transit time and blood pressure value data and acquires parameters for each individual. Many subjects refer to, e.g., several tens of persons to approximately one hundred persons with no bias in attributes such as gender, an age, and a level of blood pressure.

A user sticks the blood pressure measuring device 100 to a body surface in such a way that from the viewpoint of vertically viewing a body inside from the body surface, the first electrode 101 and the second electrode 102 sandwich an artery, and the artery and the first pulse wave detecting unit 103 overlap each other, and measures blood pressure. The measured blood pressure value is output to the display unit 107. The display unit 107 may be any device by which the recognition of a user can be made, and for example, a check can be made on a screen of a personal computer (PC) wirelessly connected to the blood pressure measuring device 100 or a portable terminal wirelessly connected to the same. Further, the display unit 107 may be provided on the casing 108 on the side opposite to the side where the first electrode 101 and the second electrode 102 are provided.

Usually, when blood pressure is measured, an arm band called a cuff is put through an arm or a wrist, and blood pressure can be measured under a press by the cuff. However, in the blood pressure measuring device 100 according to the present example embodiment, a user can measure blood pressure simply by sticking the blood pressure measuring device 100 to a body surface near an artery, and thus, there is no troublesomeness in attaching the device. Further, a press by the cuff is unnecessary, and thus, blood pressure can be non-invasively measured. FIG. 6 illustrates a state when the blood pressure measuring device 100 is attached to an upper arm. FIG. 6 illustrates an example in which the blood pressure measuring device 100 is stuck over a brachial artery 2 of a left upper arm 1.

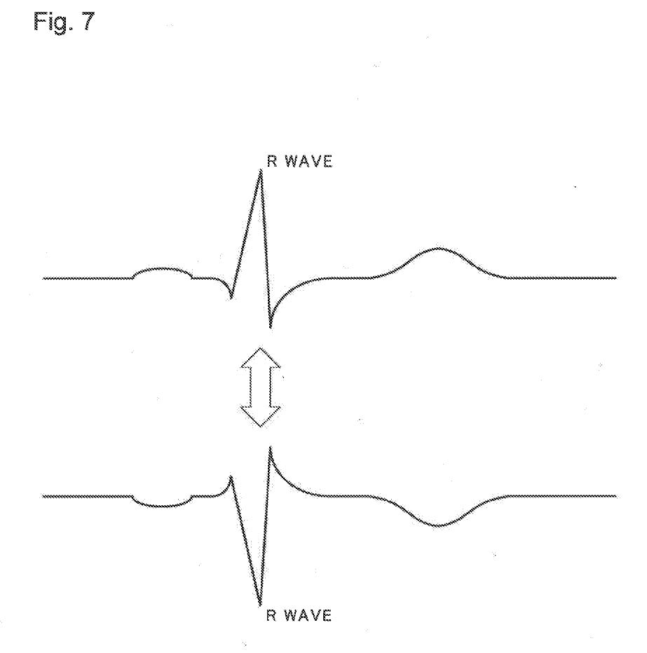

In blood pressure measurement using pulse wave transit time, generally, electrodes need to be stuck on a chest where a heart with a large signal intensity is positioned, or on both hands or feet with a large potential difference in order to measure an electrocardiogram. However, the present inventors found that a signal polarity of an electrocardiogram is inverted at a boundary where the first electrode 101 and the second electrode 102 cross over an artery, and proposes a method of measuring an electrocardiogram from one region of a body other than a chest. FIG. 7 is an image diagram illustrating that a polarity of an electrocardiogram waveform is inverted by the first electrode 101 and the second electrode 102 crossing over an artery. By using this phenomenon, a potential difference between the electrodes stuck in such a way as to sandwich an artery can be calculated, and an electrocardiogram can be measured even when a signal is very weak. At a position where a potential difference between the first electrode 101 and the second electrode 102 is the largest in a region where a polarity inversion can be observed, a signal/noise ratio (S/N ratio) is the largest, and thus, measurement is desirably performed at this location. In the present example embodiment, because measurement is performed at the brachial artery 2 of the left upper arm 1, a user searches for a position where a signal polarity is inverted, by moving the blood pressure measuring device 100 in a circumferential direction of an arm. An electrocardiogram waveform as illustrated in FIG. 7 is displayed on the display unit 107, and thereby, inversion occurrence is easily recognized and positioning is easy.

Note that at a location where the first electrode 101 and the second electrode 102 largely deviate from an artery, a direction to move the electrodes is not recognized in some cases. However, when pulse waves are also measured by the first pulse wave detecting unit 103 and the pulse wave measuring unit 105, a direction of increasing a potential difference may be found by moving the first and second electrodes in a direction of increasing measured values of pulse waves, and the optimum measurement position is easily found.

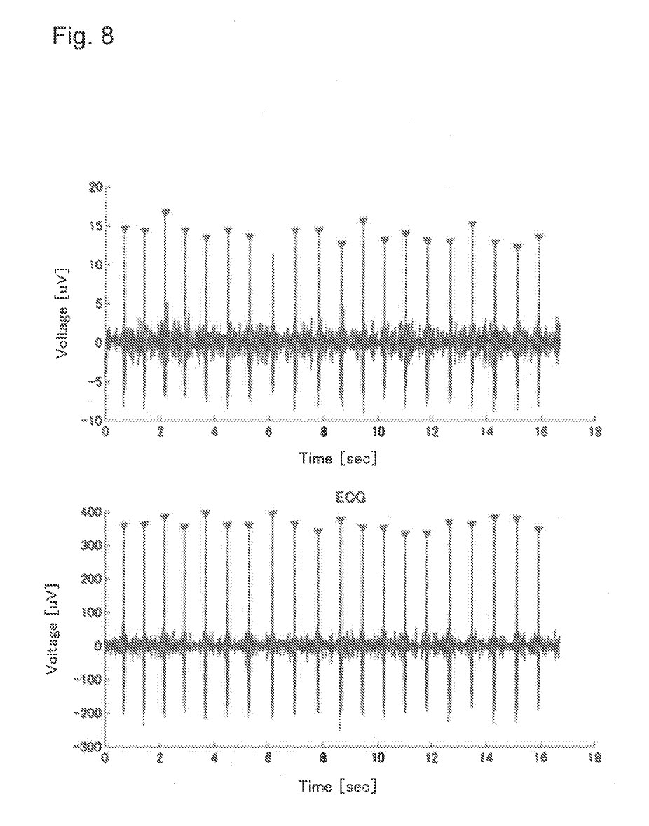

FIG. 8 illustrates electrocardiograms measured by the method of the present example embodiment and the existing method (the method described above in the Background Art, in which a plurality of sensors need to be stuck to a plurality of regions of a body). The method of the present example embodiment corresponds to an upper side in FIG. 8, and the existing method corresponds to a lower side in FIG. 8. Note that “ECG” in FIG. 8 is an abbreviation for electrocardiogram. In comparison of R waves of the electrocardiograms measured by these two methods, it is understood that the present example embodiment can measure a potential difference of approximately 1/20 of that in the existing method, and can measure a very weak electrocardiogram. Downward triangles in the drawing designate peaks of the R waves.

Further, in the present example embodiment, the electrodes may be stuck in such a way as to be adjusted to an artery position from a body surface. For this reason, unlike PTL 2, many electrodes are unnecessary, and only the two electrodes suffice. While increase in the number of electrodes leads to longer processing time for finding an electrocardiogram signal of the maximum amplitude, the number of electrodes is two in the present example embodiment, and thus, the signal processing time can be shortened. Furthermore, since electrodes do not need to be stuck to many regions such as a chest, both hands, and both feet, and the electrodes may be stuck to one region (an upper arm in the present example embodiment), an attaching load for a user is small, and both hands are not restrained. In addition, since only the two electrodes are stuck, a user without expertise can easily measure an electrocardiogram.

Note that when this blood pressure measuring device 100 is used during motion, it is expected that artifacts (noises mixed in a pulse wave and an electrocardiogram) such as vibration due to the motion and an electromyogram due to muscle at a position to which electrodes are stuck are generated, and a pulse waveform and an electrocardiogram waveform are disturbed. However, addition of a process of detecting this disturbance of the waveforms and suspending measurement during motion can avoid erroneous detection, suppress power consumption, and increase usable time. Further, mounting an acceleration sensor or the like capable of detecting a motion state of a user enables more accurate detection of whether or not motion is made.

### Second Example Embodiment

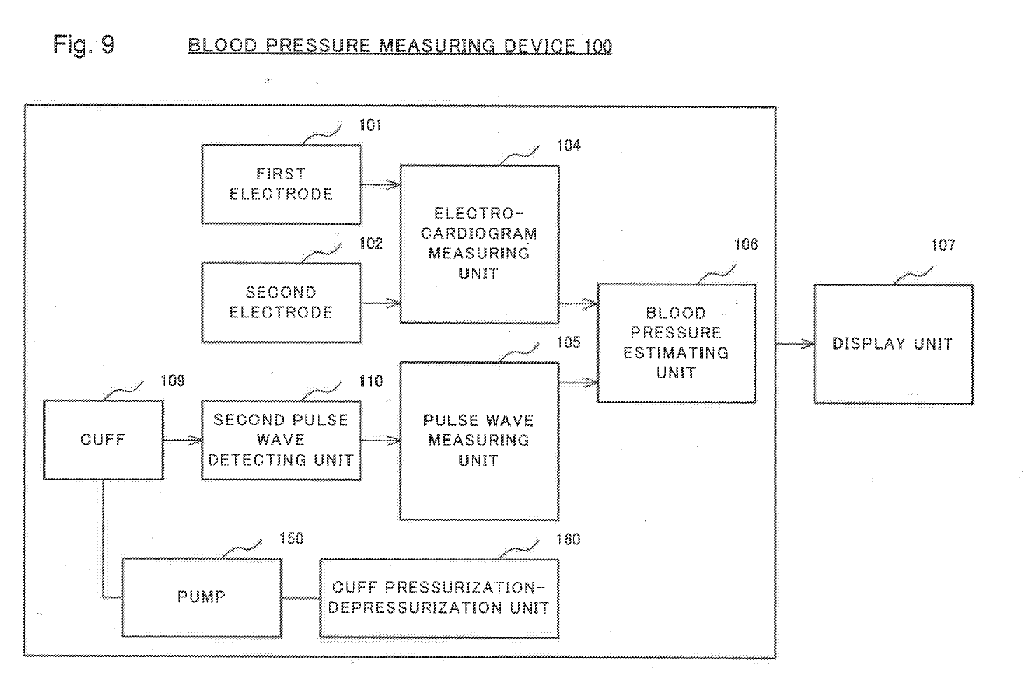

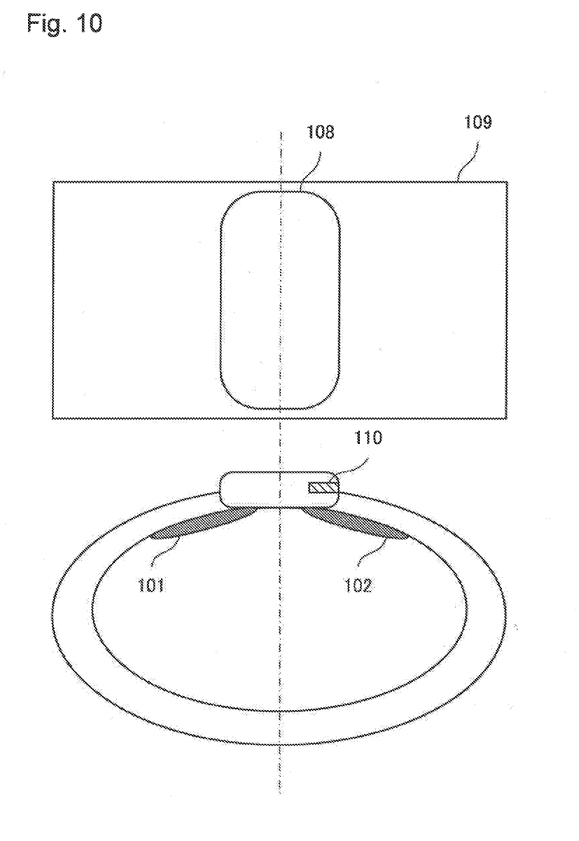

FIG. 9 is a block diagram in a second example embodiment of a blood pressure measuring device according to the present invention. A difference from the first example embodiment is that a cuff 109 is used for acquiring a pulse wave. The blood pressure measuring device 100 includes the first electrode 101, the second electrode 102, the cuff 109, a second pulse wave detecting unit 110 connected to the cuff 109, the electrocardiogram measuring unit 104, the pulse wave measuring unit 105, and the blood pressure estimating unit 106. Further, the blood pressure measuring device 100 includes a pump 150 for sending air to the cuff 109, and a cuff pressurization-depressurization unit 160 that performs pressurization and depressurization to the cuff. FIG. 10 illustrates a plan view and a cross-sectional view of the blood pressure measuring device 100. The second pulse wave detecting unit 110 connected to the cuff 109 is preferably a sensor of which type is any one of a vibration sensor, a pressure sensor, and a piezoelectric sensor. Piping is made in such a way that the pressure sensor can measure internal pressure of the cuff 109. Specifically, the pressure sensor is connected to an air pipe (not illustrated) that sends air to the cuff 109. The vibration sensor and the piezoelectric sensor are installed between the cuff 109 and a body surface. In addition, the first electrode 101 and the second electrode 102 are arranged in a longitudinal direction of the cuff 109. In FIG. 10, it is assumed that the pressure sensor is used.

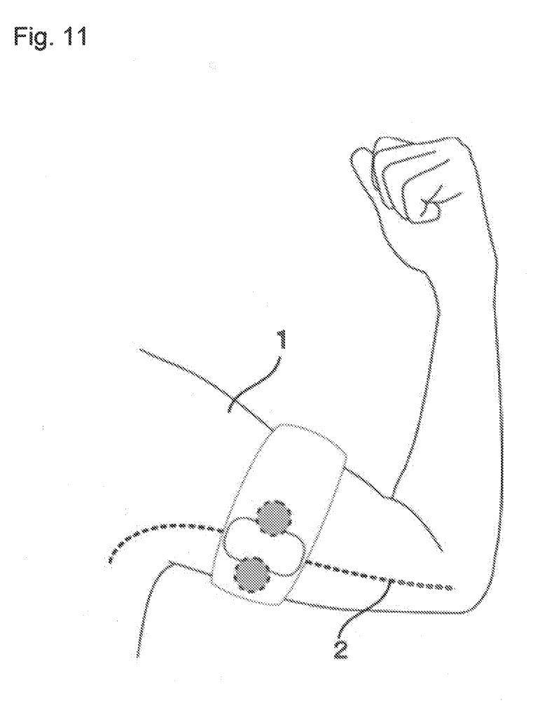

FIG. 11 illustrates a state when the blood pressure measuring device 100 is attached. Also in the present example embodiment, the blood pressure measuring device 100 is attached to an upper arm portion. After the blood pressure measuring device 100 is attached, a signal is measured by the second pulse wave detecting unit 110 while air is supplied to the cuff 109, and at timing when pulsation of an artery is detected, that is, a pulse wave output equal to or larger than any value is detected, supply of air to the cuff 109 is stopped. After that, adjustment of the air pressure is desirably continued in such a way that a pulse wave can be detected. In FIG. 10, the cuff 109 is assumed to have a shape covering an entire attachment region, but may have a shape covering only a part of the attachment region as long as no significant positional deviation occurs after attachment, and may have a shape enabling the second pulse wave detecting unit to detect pulsation of an artery.

Using the cuff enables the sensor to contact with a body surface at appropriate pressure. Thus, a signal with a high S/N ratio and a less body motion noise can be acquired, and further, positional deviation of the blood pressure measuring device 100 can be suppressed. Since the cuff is used in the present example embodiment, surfaces belonging to the first electrode 101 and the second electrode 102 and contacting with a body surface do not need to have an adhesive property.

### Third Example Embodiment

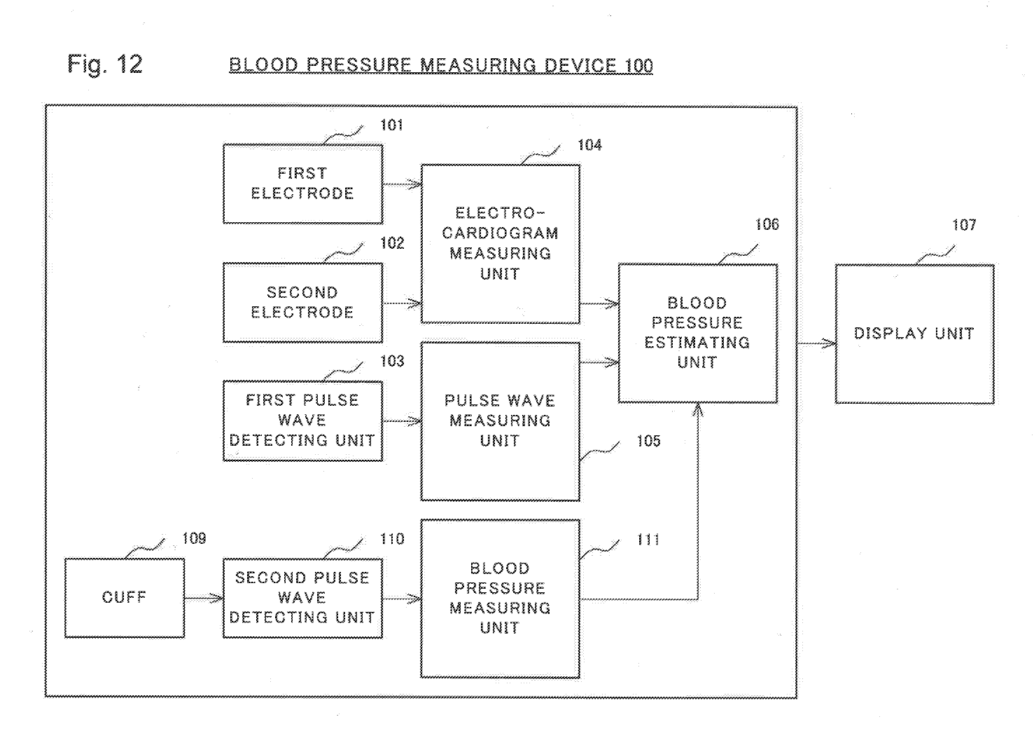

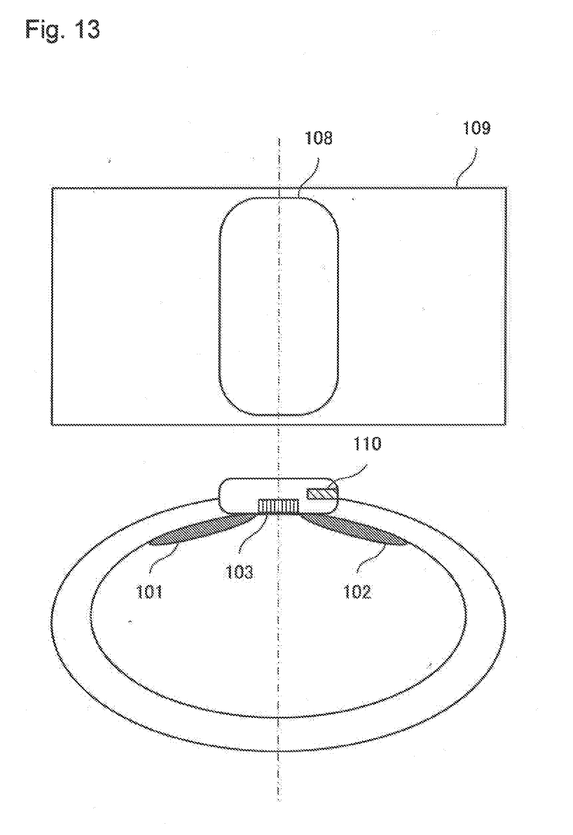

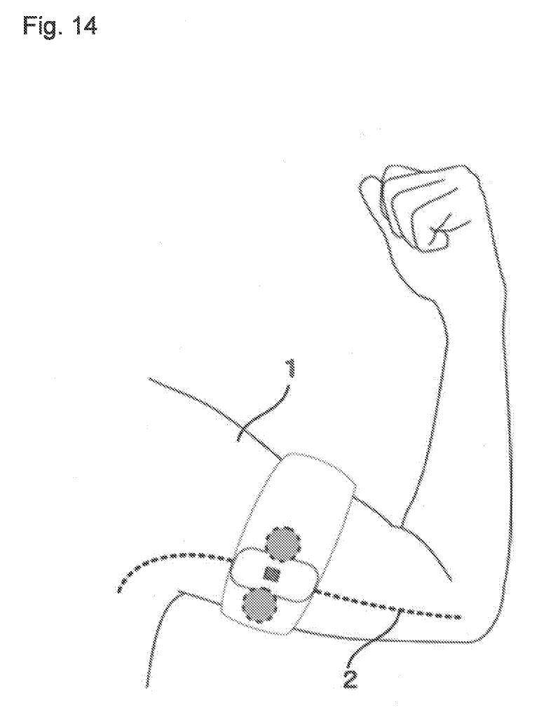

FIG. 12 illustrates a block diagram of a blood pressure measuring device according to a third example embodiment of the present invention. The present example embodiment differs from the second example embodiment in having a calibration function by the cuff 109. In FIG. 12, illustrations of the pump and the cuff pressurization-depressurization unit are omitted. The blood pressure measuring device 100 includes the first electrode 101, the second electrode 102, the first pulse wave detecting unit 103, the cuff 109, the second pulse wave detecting unit 110 connected to the cuff 109, the electrocardiogram measuring unit 104, the pulse wave measuring unit 105, a blood pressure measuring unit 111, and the blood pressure estimating unit 106. FIG. 13 illustrates a plan view and a cross-sectional view of the blood pressure measuring device 100. The blood pressure measuring unit 111 calculates diastolic blood pressure and systolic blood pressure, based on a pressurized pulse wave acquired from a press by the cuff 109, and sends the calculated pressure to the blood pressure estimating unit 106. Based on the blood pressure values sent from the blood pressure measuring unit 111, and pulse wave transit time acquired from an electrocardiogram and pulse waves sent from the electrocardiogram measuring unit 104 and the pulse wave measuring unit 105, the blood pressure estimating unit 106 derives the above-described relation formula between pulse wave transit time and blood pressure. Specifically, the blood pressure estimating unit 106 calculates α and β in the above-described formula (1).

First, a user wears the blood pressure measuring device 100 in such a way that an artery and the first pulse wave detecting unit 103 overlap each other, and the first electrode 101 and the second electrode 102 sandwich the artery, and measures diastolic blood pressure and systolic blood pressure by the blood pressure measuring method using a press by the cuff 109. FIG. 14 illustrates a state where the blood pressure measuring device 100 is attached. Also in the present example embodiment, with the cuff 109 being wound around a left upper arm, measurement is performed. The blood pressure measurement method used at this time is a well-known oscillometric method, and for example, the cuff 109 is pressurized until pressure thereof becomes equal to or higher than systolic blood pressure, and then, systolic blood pressure and diastolic blood pressure are measured while the cuff is depressurized, and after the measurement, the cuff is completely depressurized. Simultaneously with, before, or after this blood pressure measurement, pulse wave transit time is measured by using the first electrode 101, the second electrode 102, the first pulse wave detecting unit 103, the electrocardiogram measuring unit 104, and the pulse wave measuring unit 105. Then, based on the measured systolic blood pressure value and pulse wave transit time, calibration is performed on the parameters in the above-described relation formula between systolic blood pressure and pulse wave transit time for calculating a blood pressure estimated value, the relation formula being held by the blood pressure estimating unit 106. After the calibration, systolic blood pressure is estimated by using the relation formula between systolic blood pressure and pulse wave transit time.

Note that the calibration is performed for each user. The calibration may be periodically repeated.

Further, by using the blood pressure data acquired at the time of the calibration, not only systolic blood pressure but also diastolic blood pressure can be estimated. Generally, fluctuation in systolic blood pressure and diastolic blood pressure is smaller than fluctuation in pulse pressure (=“systolic blood pressure”−“diastolic blood pressure”). By using the cuff 109, not only systolic blood pressure but also diastolic blood pressure are measured at the time of the calibration, and a formula acquired by replacing the parameters α and β of the above-described formula (1) respectively with γ and δ that are parameters for diastolic blood pressure is also generated. Thereby, depending on fluctuation in estimated systolic blood pressure, an estimated value of diastolic blood pressure can be calculated.

Besides, diastolic blood pressure can be estimated from a relation between change in a blood pressure value and change in a pulse waveform acquired by the first pulse wave detecting unit 103. This is the matter that fluctuation in a blood pressure value is related to, for example, a pulse wave amplitude that is one parameter of a pulse waveform, and when an amplitude of a pulse wave increases, diastolic blood pressure is also increased to that extent, and when an amplitude decreases, diastolic blood pressure is also decreased.

Further, since the cuff 109 is provided in the present example embodiment, an existing oscillometric method or the like may be used in measuring diastolic blood pressure. In other words, diastolic blood pressure may be measured at the stage of pressurizing the cuff, and then, systolic blood pressure may be measured by the method of the present example embodiment.

Furthermore, although the first pulse wave detecting unit 103 and the second pulse wave detecting unit 110 are described as separate constituents in the present example embodiment, the first pulse wave detecting unit 103 and the second pulse wave detecting unit 110 may be integrated.

### Fourth Example Embodiment

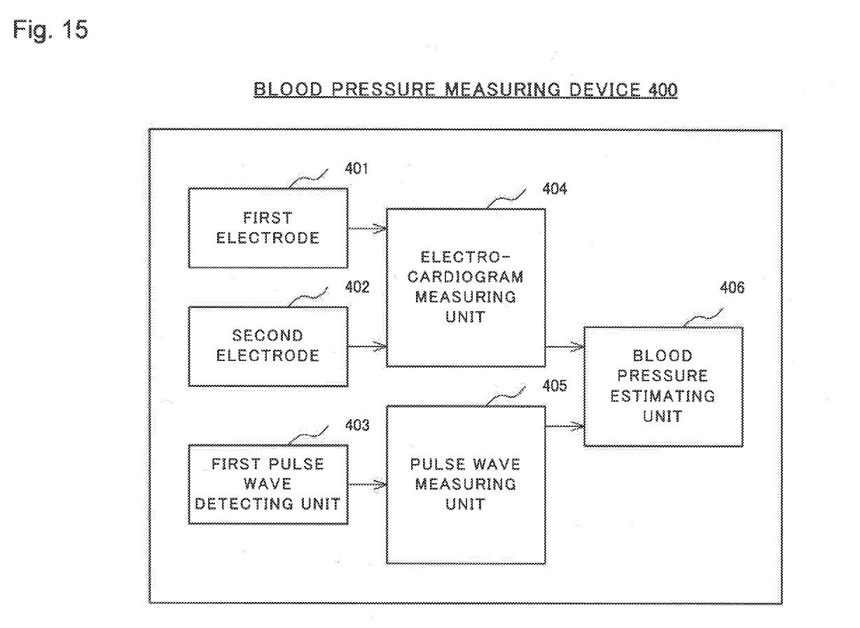

FIG. 15 is a block diagram illustrating a blood pressure measuring device 400 according to a fourth example embodiment of the present invention.

The blood pressure measuring device 400 includes a first electrode 401 and a second electrode 402 that are made to contact with a body surface near an artery of an upper arm or the like. An electrocardiogram measuring unit 404 measures a potential difference between the first electrode 401 and the second electrode 402, and acquires a first time at which at least a predetermined portion in an electrocardiogram occurs. A first pulse wave detecting unit 403 acquires pulse wave information from a body surface near an artery. From this pulse wave information, a pulse wave measuring unit 405 acquires a second time at which a predetermined portion in the pulse wave occurs. A blood pressure estimating unit 406 calculates pulse wave transit time from a difference between the first time 401 and the second time 402, and calculates estimated blood pressure, based on the pulse wave transit time and the predefined relation between pulse wave transit time and a blood pressure value that are described in the first example embodiment.

Thereby, blood pressure can be measured simply by easy attachment to one region of a body.

### Other Example Embodiment

Although an electrocardiogram waveform is measured at a brachial artery in the first to fourth example embodiments, measurement may be performed at least one of a carotid artery, a superficial temporal artery, a facial artery, a radial artery, a femoral artery, a popliteal artery, a posterior tibial artery, and a dorsalis pedis artery other than a brachial artery.

A person himself/herself who measures with the blood pressure measuring device 100 is also assumed to be a user in the first to fourth example embodiments, but is not limited to this, and includes a doctor, a nurse, a caregiver, a family member, and the like. Although calibration by the cuff 109 and the blood pressure measuring unit 111 included in the blood pressure measuring device 100 is used in the third example embodiment, but a blood pressure value of a different blood pressure gauge may be used. Although the two electrodes for electrocardiogram measurement and the one pulse wave detecting unit for pulse wave measurement are used in the first and third example embodiments, the number of electrodes and pulse wave detecting units may be increased in such a way that positional deviation during use can be addressed.

Although an R wave is used as a predetermined portion in an electrocardiogram in the first to fourth example embodiments, an electrocardiogram includes a P wave, a T wave, and a U wave as well, other than an R wave, and these waves can be used. Although a rise time of a pulse wave is used as the pulse wave information in the first to third example embodiments, other information such as peak time may be used. A parameter is only required to define a relation between pulse wave transit time and systolic blood pressure as in the formula (1).

Further, the blood pressure measuring device according to the first to fourth example embodiments may be implemented by a dedicated device, but can also be implemented by a computer (information processing device). In this case, the computer reads a software program stored in a memory (not illustrated) out to a central processing unit (CPU, not illustrated), executes the read software program in the CPU, and thereby outputs the executed result to a display unit, for example. In the cases of the above-described respective example embodiments, the software program is only required to include statements that can implement functions of respective means of the first pulse wave detecting unit 103, the electrocardiogram measuring unit 104, the pulse wave measuring unit 105, the blood pressure estimating unit 106, the cuff pressurization-depressurization unit 160, and the blood pressure measuring unit 111 illustrated in FIG. 1, FIG. 9, FIG. 12, and FIG. 15.

However, it is also supposed that the respective means appropriately include hardware. In such a case, the software program (computer program) can be regarded as constituting the present invention. Further, a computer-readable storage medium having the software program stored therein can be regarded as constituting the present invention, as well.

A part or all of the above-described example embodiments can be described also as in Supplementary Notes below, but are not limited thereto.

### (Supplementary Note 1)

A blood pressure measuring device including: a first electrode and a second electrode that are made to contact with a body surface near an artery; an electrocardiogram measuring unit that measures a potential difference between the first electrode and the second electrode, and acquires a first time at which at least a predetermined portion in an electrocardiogram occurs; a pulse wave detecting unit that detects pulse wave information from a body surface near the artery; a pulse wave measuring unit that acquires, from the pulse wave information, a second time at which a predetermined portion in a pulse wave occurs; and a blood pressure estimating unit that calculates pulse wave transit time from the first time and the second time, and calculates estimated blood pressure, based on the pulse wave transit time and a predefined relation between pulse wave transit time and a blood pressure value.

### (Supplementary Note 2)

The blood pressure measuring device according to Supplementary Note 1, wherein polarities of signals acquired by the first electrode and the second electrode are inverted from each other.

### (Supplementary Note 3)

The blood pressure measuring device according to Supplementary Note 1 or 2, further including a cuff that gives pressure to an attachment region, wherein the pulse wave detecting unit is connected to the cuff, and detects the pulse wave information.

### (Supplementary Note 4)

The blood pressure measuring device according to Supplementary Note 3, wherein the blood pressure estimating unit calculates the blood pressure value, based on a pressurized pulse wave acquired by the pulse wave detecting unit under a press by the cuff.

### (Supplementary Note 5)

The blood pressure measuring device according to any one of Supplementary Notes 1 to 4, wherein surfaces of the first electrode and the second electrode have an adhesive property.

### (Supplementary Note 6)

The blood pressure measuring device according to any one of Supplementary Notes 1 to 5, wherein the first pulse wave detecting unit and the second pulse wave detecting unit are each constituted of at least one of a vibration sensor, a pressure sensor, a piezoelectric sensor, an optical sensor, an ultrasonic sensor, a radio wave sensor, an electrostatic capacity sensor, an electric field sensor, and a magnetic field sensor.

### (Supplementary Note 7)

The blood pressure measuring device according to any one of Supplementary Notes 1 to 6, wherein the blood pressure estimating unit includes a relation formula, between the pulse wave transit time and the blood pressure value, calculated by any one of a method by statistical analysis based on the pulse wave transit time and the blood pressure values acquired from a plurality of subjects, and a method by calibration based on the pulse wave transit time and the blood pressure value acquired for each individual.

### (Supplementary Note 8)

The blood pressure measuring device according to Supplementary Note 7, wherein, when the pulse wave transit time is PWTT, systolic blood pressure is SBPest, and α and β are parameters acquired by the calibration, the relation formula is a relation formula expressed by:

SBPest=α×PWTT+β.

### (Supplementary Note 9)

The blood pressure measuring device according to any one of Supplementary Notes 1 to 8, wherein the artery is at least one of a brachial artery, a carotid artery, a superficial temporal artery, a facial artery, a radial artery, a femoral artery, a popliteal artery, a posterior tibial artery, and a dorsalis pedis artery.

### (Supplementary Note 10)

The blood pressure measuring device according to any one of Supplementary Notes 1 to 9, wherein the blood pressure estimating unit updates a relation formula between the pulse wave transit time and the blood pressure value, based on the blood pressure value acquired by the blood pressure measuring unit and the pulse wave transit time acquired from the electrocardiogram and the pulse wave being acquired by the electrocardiogram measuring unit and the pulse wave measuring unit.

### (Supplementary Note 11)

The blood pressure measuring device according to any one of Supplementary Notes 1 to 10, wherein the predetermined portion in the electrocardiogram is a specific wave in the electrocardiogram.

### (Supplementary Note 12)

The blood pressure measuring device according to Supplementary Note 11, wherein the specific wave is an R wave.

### (Supplementary Note 13)

The blood pressure measuring device according to any one of Supplementary Notes 1 to 12, wherein the second time is a rise time of a pulse wave.

### (Supplementary Note 14)

The blood pressure measuring device according to any one of Supplementary Notes 1 to 13, wherein the first electrode and the second electrode are curved in such a way as to conform to a shape of an attachment region.

### (Supplementary Note 15)

A blood pressure measuring method including: making a first electrode and a second electrode in contact with a body surface near an artery; measuring a potential difference between the first electrode and the second electrode, and acquiring a first time at which at least a predetermined portion in an electrocardiogram occurs; detecting pulse wave information from a body surface near the artery; acquiring, from the pulse wave information, a second time at which a predetermined portion in the pulse wave occurs; and calculating pulse wave transit time from the first time and the second time, and calculating estimated blood pressure, based on the pulse wave transit time and a predefined relation between pulse wave transit time and a blood pressure value.

### (Supplementary Note 16)

A blood pressure measuring program that causes a computer to execute: processing of measuring a potential difference between a first electrode and a second electrode made to contact with a body surface near an artery, and acquiring a first time at which at least a predetermined portion in an electrocardiogram occurs; processing of detecting pulse wave information from a body surface near the artery; processing of acquiring, from the pulse wave information, a second time at which a predetermined portion in the pulse wave occurs; and processing of calculating pulse wave transit time from the first time and the second time, and calculating estimated blood pressure, based on the pulse wave transit time and a predefined relation between pulse wave transit time and a blood pressure value.

The present invention is described above by citing the above-described example embodiments as model examples. However, the present invention is not limited to the above-described example embodiments. In other words, in the present invention, various aspects that can be understood by those skilled in the art can be applied within the scope of the present invention.

The present application claims priority based on Japanese patent application No. 2016-172555 filed on Sep. 5, 2016, the disclosure of which is incorporated herein in its entirety.

### REFERENCE SIGNS LIST

1 Left upper arm
2 Brachial artery
100 Blood pressure measuring device
101, 401 First electrode
102, 402 Second electrode
103, 403 First pulse wave detecting unit
104, 404 Electrocardiogram measuring unit
105, 405 Pulse wave measuring unit
106, 406 Blood pressure estimating unit
107 Display unit
108 Casing
109 Cuff
110 Second pulse wave detecting unit
111 Blood pressure measuring unit

What is claimed is:

1. A blood pressure measuring device comprising:
a first electrode and a second electrode that are made to contact with a body surface near an artery;
an electrocardiogram measuring unit that measures a potential difference between the first electrode and the second electrode, and acquiring a first time at which at least a predetermined portion in an electrocardiogram occurs;
a pulse wave detecting unit that detects pulse wave information from a body surface near the artery;
a pulse wave measuring unit that acquires, from the pulse wave information, a second time at which a predetermined portion in a pulse wave occurs; and
a blood pressure estimating unit that calculates pulse wave transit time from the first time and the second time, and calculating estimated blood pressure, based on the pulse wave transit time and a predefined relation between pulse wave transit time and a blood pressure value.

2. The blood pressure measuring device according to claim 1, wherein polarities of signals acquired by the first electrode and the second electrode are inverted from each other.

3. The blood pressure measuring device according to claim 1, further comprising a cuff that gives pressure to an attachment region, wherein the pulse wave detecting unit is connected to the cuff, and detects the pulse wave information.

4. The blood pressure measuring device according to claim 3, wherein the blood pressure estimating unit calculates the blood pressure value, based on a pressurized pulse wave acquired by the pulse wave detecting unit under a press by the cuff.

5. The blood pressure measuring device according to claim 1, wherein surfaces of the first electrode and the second electrode have an adhesive property.

6. The blood pressure measuring device according to claim 1, wherein the first pulse wave detecting unit and the second pulse wave detecting unit are each constituted of at least one of a vibration sensor, a pressure sensor, a piezoelectric sensor, an optical sensor, an ultrasonic sensor, a radio wave sensor, an electrostatic capacity sensor, an electric field sensor, and a magnetic field sensor.

7. The blood pressure measuring device according to claim 1, wherein the blood pressure estimating unit includes a relation formula, between the pulse wave transit time and the blood pressure value, calculated by any one of a method by statistical analysis based on the pulse wave transit time and the blood pressure values acquired from a plurality of subjects, and a method by calibration based on the pulse wave transit time and the blood pressure value acquired for each individual.

8. The blood pressure measuring device according to claim 7, wherein, when the pulse wave transit time is PWTT, systolic blood pressure is SBPest, and α and β are parameters acquired by the calibration, the relation formula is a relation formula expressed by:
SBPest=α×PWTT+β.

9. The blood pressure measuring device according to claim 1, wherein the artery is at least one of a brachial artery, a carotid artery, a superficial temporal artery, a facial artery, a radial artery, a femoral artery, a popliteal artery, a posterior tibial artery, and a dorsalis pedis artery.

10. The blood pressure measuring device according to claim 1, wherein the blood pressure estimating unit updates a relation formula between the pulse wave transit time and the blood pressure value, based on the blood pressure value acquired by the blood pressure measuring unit and the pulse wave transit time acquired from the electrocardiogram and the pulse wave being acquired by the electrocardiogram measuring unit and the pulse wave measuring unit.

11. The blood pressure measuring device according to claim 1, wherein the predetermined portion in the electrocardiogram is a specific wave in the electrocardiogram.

12. The blood pressure measuring device according to claim 11, wherein the specific wave is an R wave.

13. The blood pressure measuring device according to claim 1, wherein the second time is a rise time of a pulse wave.

14. The blood pressure measuring device according to claim 1, wherein the first electrode and the second electrode are curved in such a way as to conform to a shape of an attachment region.

15. A blood pressure measuring method comprising:
making a first electrode and a second electrode in contact with a body surface near an artery;
measuring a potential difference between the first electrode and the second electrode, and acquiring a first time at which at least a predetermined portion in an electrocardiogram occurs;
detecting pulse wave information from a body surface near the artery;
acquiring, from the pulse wave information, a second time at which a predetermined portion in the pulse wave occurs; and
calculating pulse wave transit time from the first time and the second time, and calculating estimated blood pressure, based on the pulse wave transit time and a predefined relation between pulse wave transit time and a blood pressure value.

16. A recording medium recording a blood pressure measuring program that causes a computer to execute:
processing of measuring a potential difference between a first electrode and a second electrode made to contact with a body surface near an artery, and acquiring a first time at which at least a predetermined portion in an electrocardiogram occurs;
processing of detecting pulse wave information from a body surface near the artery;
processing of acquiring, from the pulse wave information, a second time at which a predetermined portion in the pulse wave occurs; and
processing of calculating pulse wave transit time from the first time and the second time, and calculating estimated blood pressure, based on the pulse wave transit time and a predefined relation between pulse wave transit time and a blood pressure value.
